(19) United States
(12) Patent Application Publication
(10) Pub. No.: US 2018/0000418 A1
(43) Pub. Date: Jan. 4, 2018

(54) METHOD FOR DETECTING WEARABLE DEVICE, AND WEARABLE DEVICE

(71) Applicant: Huawei Technologies Co., Ltd., Shenzhen, Guangdong (CN)

(72) Inventors: Liang LI, Shenzhen (CN); Zexu QIAN, Shenzhen (CN)

(21) Appl. No.: 15/541,469

(22) PCT Filed: Jan. 5, 2015
(86) PCT No.: PCT/CN2015/070093

§ 371 Date: Jul. 3, 2017

(51) Int. Cl. A61B 5/00; A61B 5/0205; A61B 5/0285; A61B 5/024; A61B 5/145; A61B 5/021

(52) U.S. Cl. CPC A61B 5/6844; A61B 5/681; A61B 5/6803; A61B 5/02055; A61B 5/021; A61B 5/02438; A61B 5/14532; A61B 5/0285

(57) ABSTRACT

Embodiments of the present invention disclose a method for detecting a wearable device, and the wearable device, where the method includes: detecting a value of a distance between the wearable device and a user; determining whether the value of the distance between the wearable device and the user exceeds a preset distance threshold; if the value of the distance between the wearable device and the user does not exceed the preset distance threshold, detecting a body feature value of the user within a preset time period; and if the body feature value of the user does not exceed a preset threshold range of the body feature value of the user, determining that the user has worn the wearable device.

### CROSS-REFERENCE TO RELATED APPLICATIONS

This application is a National Stage of International Application No. PCT/CN2015/070093, filed on Jan. 5, 2015, which is hereby incorporated by reference in its entirety.

### TECHNICAL FIELD

The present invention relates to the communications field, and in particular, to a method for detecting a wearable device, and the wearable device.

### BACKGROUND

A wearable device is a portable device that is worn directly on a body or is integrated with a user's clothing and accessory. The wearable device is a hardware device that uses software support, data interaction, and cloud interaction to implement various functions. In the prior art, the following two manners are mainly used to detect whether the wearable device has been worn on the user's body. One is to use a capacitive sensor to perform detection. However, when the capacitive sensor is in use, the capacitive sensor fails when a metal object is close to the capacitive sensor, and a detection success rate is low. The other is to use a visible light sensor to perform detection. However, the visible light sensor cannot identify whether a human body is close to the wearable device or another object is close to the wearable device, and a detection success rate is also low. If it cannot be determined whether the wearable device has been worn on the user's body, the wearable device cannot be used, thereby causing inconvenience to the user.

### SUMMARY

Embodiments of the present invention provide a method for detecting a wearable device, and the wearable device. In the method, whether a user has worn the wearable device can be determined quickly and accurately by detecting a value of a distance between the wearable device and the user, and a body feature value of the user.

According to a first aspect, the embodiments of the present invention provide a method for detecting a wearable device, including:

detecting a value of a distance between the wearable device and a user;

determining whether the value of the distance between the wearable device and the user exceeds a preset distance threshold;

if the value of the distance between the wearable device and the user does not exceed the preset distance threshold, detecting a body feature value of the user; and

if the body feature value of the user does not exceed a preset threshold range of the body feature value of the user, determining that the user has worn the wearable device.

With reference to the first aspect, in a first possible implementation manner of the first aspect, after the determining that the user has worn the wearable device, the method further includes:

setting a preset time period; and

detecting the body feature value of the user according to the set preset time period.

With reference to the first possible implementation manner of the first aspect, in a second possible implementation manner of the first aspect, the preset time period is set by the user.

With reference to the first aspect, in a third possible implementation manner of the first aspect, the method further includes:

if the value of the distance between the wearable device and the user exceeds the preset distance threshold, determining that the wearable device is not worn by the user.

With reference to the first aspect, in a fourth possible implementation manner of the first aspect, the method further includes:

if the body feature value of the user exceeds a preset threshold of the body feature value of the user, determining that the wearable device is not worn by the user.

With reference to any one of the first aspect to the fourth possible implementation manner of the first aspect, in a fifth possible implementation manner of the first aspect,

the body feature value of the user includes one or more of a temperature value, a heart rate value, a blood glucose value, a blood pressure value, or a blood flow velocity value.

With reference to any one of the first aspect to the third possible implementation manner of the first aspect, in a sixth possible implementation manner of the first aspect,

the value of the distance between the wearable device and the user is obtained by using a distance sensor.

With reference to any one of the first aspect to the fourth possible implementation manner of the first aspect, in a seventh possible implementation manner of the first aspect,

the body feature value of the user is obtained by using a human body feature sensor.

According to a second aspect, the embodiments of the present invention further provide a wearable device, including: a distance sensor, a human body feature sensor, and a processor, where

the distance sensor is configured to detect a value of a distance between the wearable device and a user;

the human body feature sensor is configured to detect a body feature value of the user; and

the processor is configured to: determine whether the value of the distance between the wearable device and the user exceeds a preset distance threshold, if the value of the distance between the wearable device and the user does not exceed the preset distance threshold, control the human body feature sensor to detect the body feature value of the user, and if the body feature value of the user does not exceed a preset threshold range of the body feature value of the user, determine that the user has worn the wearable device.

With reference to the second aspect, in a first possible implementation manner of the second aspect, the processor is further configured to:

set a preset time period; and

detect the body feature value of the user according to the set preset time period.

With reference to the first possible implementation manner of the second aspect, in a second possible implementation manner of the second aspect, the preset time period is set by the user.

With reference to the second aspect, in a third possible implementation manner of the second aspect, the processor is further configured to:

if the value of the distance between the wearable device and the user exceeds the preset distance threshold, determine that the wearable device is not worn by the user.

With reference to the second aspect, in a fourth possible implementation manner of the second aspect, the processor is further configured to:

if the body feature value of the user exceeds a preset threshold of the body feature value of the user, determine that the wearable device is not worn by the user.

With reference to any one of the second aspect to the fourth possible implementation manner of the second aspect, in a fifth possible implementation manner of the second aspect,

the body feature value of the user includes one or more of a temperature value, a heart rate value, a blood glucose value, a blood pressure value, or a blood flow velocity value.

According to the method for detecting a wearable device, and the wearable device in the embodiments of the present invention, a value of a distance between the wearable device and a user may be first detected, then if the value of the distance between the wearable device and the user does not exceed a preset distance threshold, whether a body feature value of the user exceeds a threshold range is detected, and if the body feature value of the user does not exceed the threshold range, that the user has worn the wearable device is determined. That the user has worn the wearable device is determined by combining the value of the distance between the wearable device and the user and the body feature value of the user, so that whether the wearable device is worn on a human body can be identified accurately, and detection accuracy is high. In addition, in this embodiment of the present invention, the value of the distance between the wearable device and the user is first detected, and the body feature value is not detected at the same time, thereby avoiding that a detection time is prolonged when the value of the distance and the body feature value are detected at the same time. Moreover, if the value of the distance between the wearable device and the user exceeds the preset distance threshold, there is no need to detect the body feature value of the user, thereby optimizing a detection process. Therefore, compared with the prior art, in the embodiments of the present invention, whether the user has worn the wearable device can be determined more quickly and more accurately.

### BRIEF DESCRIPTION OF DRAWINGS

To describe the technical solutions in the embodiments of the present invention more clearly, the following briefly describes the accompanying drawings required for describing the embodiments. Apparently, the accompanying drawings in the following description show merely some embodiments of the present invention, and a person of ordinary skill in the art may still derive other drawings from these accompanying drawings without creative efforts.

FIG. 1 is a schematic structural diagram of a smart watch according to an embodiment of the present invention;

FIG. 2 is a schematic structural diagram of smart glasses according to an embodiment of the present invention;

FIG. 3 is a schematic structural diagram of a smart band according to an embodiment of the present invention;

FIG. 4 is a schematic flowchart of a method for detecting a wearable device according to a first embodiment of the present invention;

FIG. 5 is a schematic flowchart of a method for detecting a wearable device according to a second embodiment of the present invention; and

FIG. 6 is a schematic structural diagram of a wearable device according to an embodiment of the present invention.

### DESCRIPTION OF EMBODIMENTS

The following clearly describes the technical solutions in the embodiments of the present invention with reference to the accompanying drawings in the embodiments of the present invention. Apparently, the described embodiments are a part rather than all of the embodiments of the present invention. All other embodiments obtained by a person of ordinary skill in the art based on the embodiments of the present invention without creative efforts shall fall within the protection scope of the present invention.

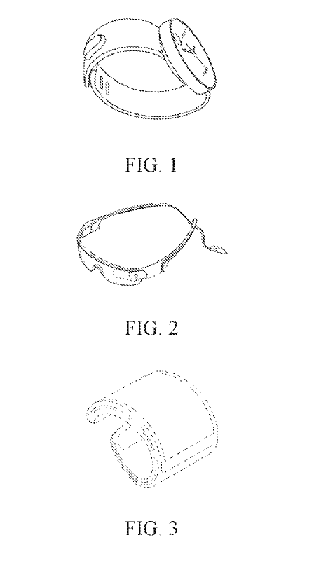

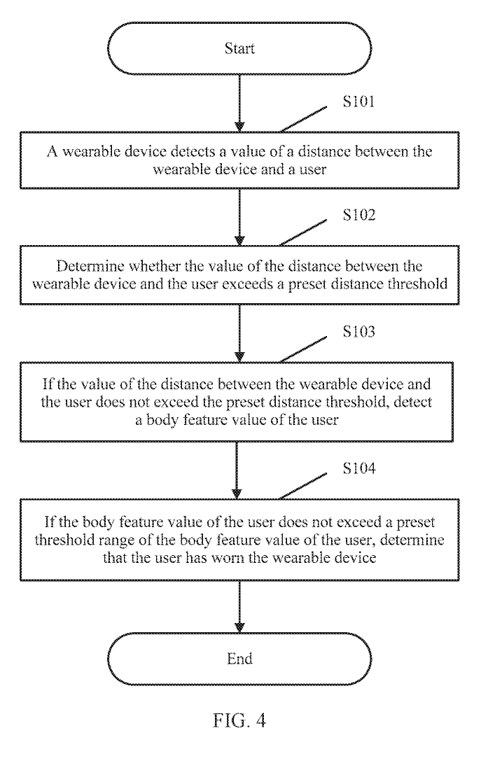

Referring to FIG. 1, FIG. 2, and FIG. 3, during specific implementation, a wearable device described in the embodiments of the present invention may include: a smart watch, a smart band, a smart ring, smart glasses, and the like. The foregoing wearable devices are merely exemplary rather than exhaustive. The wearable device includes but is not limited to the foregoing wearable devices.

Referring to FIG. 4, FIG. 4 is a schematic flowchart of a method for detecting a wearable device according to a first embodiment of the present invention, the method specifically includes the following steps.

S101. The wearable device detects a value of a distance between the wearable device and a user.

Specifically, the wearable device detects the value of the distance between the wearable device and the user by using a distance sensor, and determines, by using the value of the distance between the wearable device and the user, whether the user wears the wearable device. It should be understood that, the distance sensor in this embodiment of the present invention includes an optical proximity sensor, a linear proximity sensor, an ultrasonic displacement sensor, and the like.

S102. Determine whether the value of the distance between the wearable device and the user exceeds a preset distance threshold.

Specifically, the wearable device obtains the value of the distance between the wearable device and the user, and determines whether the value is less than or equal to the preset distance threshold. It should be understood that, a value range of the preset distance threshold is greater than or equal to 0 cm, and is less than or equal to 2 cm. For example, if the value, obtained by the wearable device, of the distance between the wearable device and the user is 1.5 cm, the value is less than the preset distance threshold. For another example, if the value, obtained by the wearable device, of the distance between the wearable device and the user is 2 cm, the value is equal to the preset distance threshold. For another example, if the value, obtained by the wearable device, of the distance between the wearable device and the user is 2.5 cm, the value is greater than the preset distance threshold.

S103. If the value of the distance between the wearable device and the user does not exceed the preset distance threshold, detect a body feature value of the user.

Specifically, when the value of the distance between the wearable device and the user does not exceed the preset distance threshold, the wearable device obtains the body feature value of the user by using a human body feature sensor. The body feature value of the user includes one or more of a temperature value, a heart rate value, a blood glucose value, a blood pressure value, or a blood flow velocity value. For example, the wearable device obtains the temperature value of the user by using a temperature sensor on the human body feature sensor, or obtains the heart rate value of the user by using a heart rate sensor on the human body feature sensor, or obtains the blood flow velocity value of the user by using the human body feature sensor. Certainly, in another implementation manner of this embodiment of the present invention, the wearable device may also obtain a body feature value such as the blood glucose value or the blood pressure value by using the human body feature sensor. These body feature values all can reflect a body status of the user, and these body feature values are easy to obtain.

S104. If the body feature value of the user does not exceed a preset threshold range of the body feature value of the user, determine that the user has worn the wearable device.

For example, a preset threshold value range of the temperature value of the user is 36° C. to 38° C., and if the temperature value of the user obtained by the wearable device by using the temperature sensor is 35° C., the body feature value of the user exceeds the preset threshold range of the body feature value of the user. For another example, a preset threshold value range of the heart rate value of the user is 60 beats to 100 beats per minute, and if the heart rate value of the user obtained by the heart rate sensor on the human body feature sensor is 75 beats per minute, the body feature value of the user does not exceed the preset threshold range of the body feature value of the user. For another example, a preset threshold of a forearm blood flow velocity of the user is 6 ml/min/100 g to 10 ml/min/100 g, and when a forearm skin blood flow velocity of the user detected by the wearable device is 8 ml/min/100 g, the body feature value of the user does not exceed the preset threshold range of the body feature value of the user. Similarly, thresholds of the blood pressure value and the blood glucose value may also be set in advance. After collecting the body feature value information, the wearable device compares the body feature value with the preset threshold to determine whether the body feature value exceeds the preset threshold range. According to an actual requirement, the wearable device may collect several body feature values of the user at the same time, or just collect one body feature value of the user.

It should be noted that, when the value of the distance between the wearable device and the user is less than or equal to the preset distance threshold, the body feature value of the user is detected. If the value of the distance between the wearable device and the user exceeds the preset distance threshold, it is directly determined that the wearable device is not worn by the user. Certainly, if the body feature value of the user exceeds the preset threshold of the body feature value of the user, it is determined that the wearable device is not worn by the user.

According to the method for detecting a wearable device, and the wearable device in this embodiment of the present invention, a value of a distance between the wearable device and a user may be first detected, then if the value of the distance between the wearable device and the user does not exceed a preset distance threshold, whether a body feature value of the user exceeds a threshold range is detected, and if the body feature value of the user does not exceed the threshold range, that the user has worn the wearable device is determined. That the user has worn the wearable device is determined by combining the value of the distance between the wearable device and the user and the body feature value of the user, so that whether the wearable device is worn on a human body can be identified accurately, and detection accuracy is high. In addition, in this embodiment of the present invention, the value of the distance between the wearable device and the user is first detected, and the body feature value is not detected at the same time, thereby avoiding that a detection time is prolonged when the value of the distance and the body feature value are detected at the same time. Moreover, if the value of the distance between the wearable device and the user exceeds the preset distance threshold, there is no need to detect the body feature value of the user, thereby optimizing a detection process. Therefore, compared with the prior art, in this embodiment of the present invention, whether the user has worn the wearable device can be determined more quickly and more accurately.

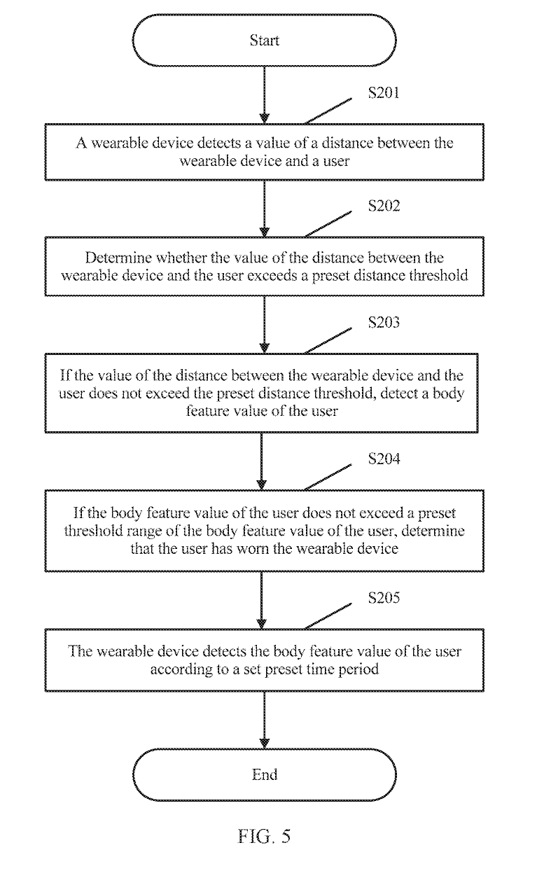

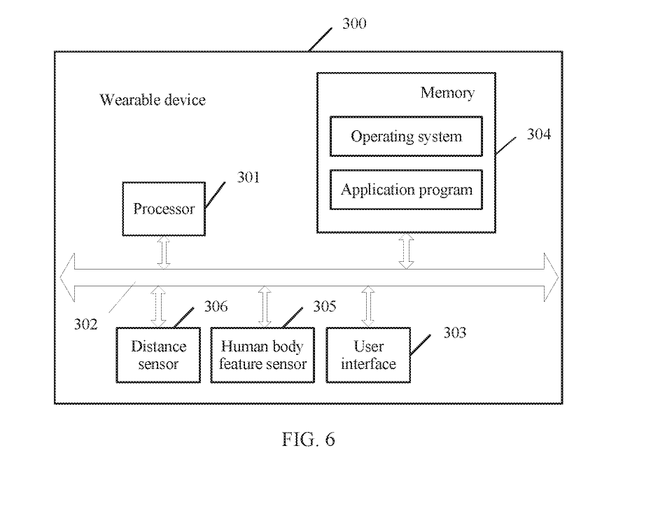

Referring to FIG. 5, FIG. 5 is a schematic flowchart of a method for detecting a wearable device according to a second embodiment of the present invention, the method specifically includes the following steps.

S201. The wearable device detects a value of a distance between the wearable device and a user.

Specifically, the wearable device detects the value of the distance between the wearable device and the user by using a distance sensor or an optical proximity sensor, and determines, by using the value of the distance between the wearable device and the user, whether the user wears the wearable device. It should be understood that, the distance sensor in this embodiment of the present invention includes the optical proximity sensor, a linear proximity sensor, an ultrasonic displacement sensor, and the like.

S202. Determine whether the value of the distance between the wearable device and the user exceeds a preset distance threshold.

Specifically, the wearable device obtains the value of the distance between the wearable device and the user, and determines whether the value is less than or equal to the preset distance threshold. It should be understood that, a value range of the preset distance threshold is greater than or equal to 0 cm, and is less than or equal to 2 cm. For example, if the value, obtained by the wearable device, of the distance between the wearable device and the user is 1.5 cm, the value is less than the preset distance threshold. For another example, if the value, obtained by the wearable device, of the distance between the wearable device and the user is 2 cm, the value is equal to the preset distance threshold. For another example, if the value, obtained by the wearable device, of the distance between the wearable device and the user is 2.5 cm, the value is greater than the preset distance threshold.

S203. If the value of the distance between the wearable device and the user does not exceed the preset distance threshold, detect a body feature value of the user.

Specifically, when the value of the distance between the wearable device and the user does not exceed the preset distance threshold, the wearable device obtains the body feature value of the user by using a human body feature sensor. The body feature value of the user includes one or more of a temperature value, a heart rate value, a blood glucose value, a blood pressure value, or a blood flow velocity value.

For example, the wearable device obtains the temperature of the user by using a temperature sensor on the human body feature sensor, or obtains the heart rate value of the user by using a heart rate sensor on the human body feature sensor, or obtains the blood flow velocity value of the user by using the human body feature sensor. Certainly, in another implementation manner of this embodiment of the present invention, the wearable device may also obtain a body feature value such as the blood glucose value or the blood pressure by using the human body feature sensor. These body feature values all can reflect a body status of the user, and these body feature values are easy to obtain.

S204. If the body feature value of the user does not exceed a preset threshold range of the body feature value of the user, determine that the user has worn the wearable device.

For example, a preset threshold value range of the temperature value of the user is 36° C. to 38° C., and if the temperature value of the user obtained by the wearable device by using the temperature sensor is 35° C., the body feature value of the user exceeds the preset threshold range of the body feature value of the user. For another example, a preset threshold value range of the heart rate value of the user is 60 beats to 100 beats per minute, and if the heart rate value of the user obtained by the heart rate sensor on the human body feature sensor is 75 beats per minute, the body feature value of the user does not exceed the preset threshold range of the body feature value of the user. For another example, a preset threshold of a forearm blood flow velocity of the user is 6 ml/min/100 g to 10 ml/min/100 g, and when a forearm skin blood flow velocity of the user detected by the wearable device is 8 ml/min/100 g, the body feature value of the user does not exceed the preset threshold range of the body feature value of the user. Similarly, thresholds of the blood pressure value and the blood glucose value may also be set in advance. After collecting the body feature value information, the wearable device compares the body feature value with the preset threshold to determine whether the body feature value exceeds the preset threshold range. According to an actual requirement, the wearable device may collect several body feature values of the user at the same time, or just collect one body feature value of the user.

It should be noted that, when the value of the distance between the wearable device and the user is less than or equal to the preset distance threshold, the body feature value of the user is detected. If the value of the distance between the wearable device and the user exceeds the preset distance threshold, it is directly determined that the wearable device is not worn by the user. Certainly, if the body feature value of the user exceeds the preset threshold of the body feature value of the user, it is determined that the wearable device is not worn by the user.

S205. The wearable device detects the body feature value of the user according to a set preset time period.

Specifically, after it is determined that the user has worn the wearable device, the user may set the preset time period, and the wearable device detects the body feature value of the user according to the set preset time period. The preset time period includes 5 minutes to 10 minutes. If the wearable device that has been worn by the user is taken off, there is no need to detect the distance between the wearable device and the user again, only the body feature value of the user needs to be detected, there is no need to detect the body feature value of the user in real time, the wearable device just needs to detect the body feature value of the user every 5 minutes to 10 minutes, and whether the user wears the wearable device can be determined quickly and accurately.

According to the method for detecting a wearable device, and the wearable device in this embodiment of the present invention, a value of a distance between the wearable device and a user may be first detected, then if the value of the distance between the wearable device and the user does not exceed a preset distance threshold, whether a body feature value of the user exceeds a threshold range is detected, and if the body feature value of the user does not exceed the threshold range, that the user has worn the wearable device is determined. That the user has worn the wearable device is determined by combining the value of the distance between the wearable device and the user and the body feature value of the user, so that whether the wearable device is worn on a human body can be identified accurately, and detection accuracy is high. In addition, in this embodiment of the present invention, the value of the distance between the wearable device and the user is first detected, and the body feature value is not detected at the same time, thereby avoiding that a detection time is prolonged when the value of the distance and the body feature value are detected at the same time. Moreover, if the value of the distance between the wearable device and the user exceeds the preset distance threshold, there is no need to detect the body feature value of the user, thereby optimizing a detection process. Therefore, compared with the prior art, in this embodiment of the present invention, whether the user has worn the wearable device can be determined more quickly and more accurately.

Referring to FIG. 6, FIG. 6 is a schematic structural diagram of a wearable device according to an embodiment of the present invention, the wearable device 300 includes: at least one processor 301, a human body feature sensor 305, and a distance sensor 306, and further includes: the at least one user interface 303, the at least one communications bus 302, and the at least one memory 304.

The processor 301 may include a CPU. The human body feature sensor 305 may include a temperature sensor, a heart rate sensor, a blood glucose sensor, a blood pressure sensor, a blood flow velocity sensor, and the like. The distance sensor 306 may include an optical proximity sensor, a linear proximity sensor, an ultrasonic displacement sensor, and the like. The communications bus 302 is configured to implement connection and communication between these components. The user interface 303 may include a display (Display) and a keyboard (Keyboard). Optionally, the user interface 303 may further include a standard wired interface and a standard wireless interface. The memory 304 may be a high-speed RAM memory, or may be a non-volatile memory (non-volatile memory), for example, at least one disk memory. Optionally, the memory 304 may be at least one storage apparatus far from the foregoing processor 301.

The memory 304 stores a group of program code, and the processor 301 invokes the program code stored in the memory 304 to perform the following operations:

the distance sensor 306 is configured to detect a value of a distance between the wearable device and a user;

the human body feature sensor 305 is configured to detect a body feature value of the user; and

the processor 301 is configured to determine whether the value of the distance between the wearable device and the user exceeds a preset distance threshold, if the value of the distance between the wearable device and the user does not exceed the preset distance threshold, control the human body feature sensor to detect the body feature value of the user, and if the body feature value of the user does not exceed a preset threshold range of the body feature value of the user, determine that the user has worn the wearable device.

Specifically, the wearable device detects the value of the distance between the wearable device and the user by using the distance sensor or the optical proximity sensor, and determines, by using the value of the distance between the wearable device and the user, whether the user wears the wearable device.

The wearable device obtains the value of the distance between the wearable device and the user, and determines whether the value is less than or equal to the preset distance threshold. It should be understood that, a value range of the preset distance threshold is greater than or equal to 0 cm, and is less than or equal to 2 cm. For example, if the value, obtained by the wearable device, of the distance between the wearable device and the user is 1.5 cm, the value is less than the preset distance threshold. For another example, if the value, obtained by the wearable device, of the distance between the wearable device and the user is 2 cm, the value is equal to the preset distance threshold. For another example, if the value, obtained by the wearable device, of the distance between the wearable device and the user is 2.5 cm, the value is greater than the preset distance threshold.

The wearable device obtains the value of the distance between the wearable device and the user, and determines whether the value is less than or equal to the preset distance threshold. It should be understood that, a value range of the preset distance threshold is greater than or equal to 0 cm, and is less than or equal to 2 cm. For example, if the value, obtained by the wearable device, of the distance between the wearable device and the user is 1.5 cm, the value is less than the preset distance threshold. For another example, if the value, obtained by the wearable device, of the distance between the wearable device and the user is 2 cm, the value is equal to the preset distance threshold. For another example, if the value, obtained by the wearable device, of the distance between the wearable device and the user is 2.5 cm, the value is greater than the preset distance threshold.

In an optional embodiment, the processor 301 may invoke the program code stored in the memory 304 to further perform the following operations:

set a preset time period; and

detect the body feature value of the user according to the set preset time period.

Specifically, after determining that the user has worn the wearable device, the wearable device detects the body feature value of the user according to the time period preset by the user. The time period preset by the user may include 5 minutes to 10 minutes. If the wearable device that has been worn by the user is taken off, there is no need to detect the distance between the wearable device and the user again, only the body feature value of the user needs to be detected, there is no need to detect the body feature value of the user in real time, the wearable device just needs to detect the body feature value of the user every 5 minutes to 10 minutes, and whether the user wears the wearable device can be determined quickly and accurately.

In an optional embodiment, the processor 301 may invoke the program code stored in the memory 304 to further perform the following operation:

the preset time period is set by the user.

In an optional embodiment, the processor 301 may invoke the program code stored in the memory 304 to further perform the following operation:

if the value of the distance between the wearable device and the user exceeds the preset distance threshold, determine that the wearable device is not worn by the user.

In an optional embodiment, the processor 301 may invoke the program code stored in the memory 304 to further perform the following operation:

if the body feature value of the user exceeds a preset threshold of the body feature value of the user, determine that the wearable device is not worn by the user.

In an optional embodiment, the processor 301 may invoke the program code stored in the memory 304 to further perform the following operation:

the body feature value of the user includes one or more of a temperature value, a heart rate value, a blood glucose value, a blood pressure value, or a blood flow velocity value.

In an optional embodiment, the processor 301 may invoke the program code stored in the memory 304 to further perform the following operation:

the value of the distance between the wearable device and the user is obtained by using the distance sensor.

In an optional embodiment, the processor 301 may invoke the program code stored in the memory 304 to further perform the following operation:

the body feature value of the user is obtained by using the human body feature sensor.

Specifically, when the value of the distance between the wearable device and the user does not exceed the preset distance threshold, the wearable device obtains the body feature value of the user by using the human body feature sensor. The body feature value of the user includes one or more of a temperature value, a heart rate value, a blood glucose value, a blood pressure value, or a blood flow velocity value. For example, the wearable device obtains the temperature value of the user by using the temperature sensor on the human body feature sensor, or obtains the heart rate value of the user by using the heart rate sensor on the human body feature sensor, or obtains the blood flow velocity value of the user by using the human body feature sensor. Certainly, in another implementation manner of this embodiment of the present invention, the wearable device may also obtain a body feature value such as the blood glucose value or the blood pressure value by using the human body feature sensor. These body feature values all can reflect a body status of the user, and these body feature values are easy to obtain.

It should be understood that, a preset threshold value range of the temperature value of the user is 36° C. to 38° C., and if the temperature value of the user obtained by the wearable device by using the temperature sensor is 35° C., the body feature value of the user exceeds the preset threshold range of the body feature value of the user. For another example, a preset threshold value range of the heart rate value of the user is 60 beats to 100 beats per minute, and if the heart rate value of the user obtained by the heart rate sensor on the human body feature sensor is 75 beats per minute, the body feature value of the user does not exceed the preset threshold range of the body feature value of the user. For another example, a preset threshold of a forearm blood flow velocity of the user is 6 ml/min/100 g to 10 ml/min/100 g, and when a forearm skin blood flow velocity of the user detected by the wearable device is 8 ml/min/100 g, the body feature value of the user does not exceed the threshold range of the body feature value of the user. Similarly, thresholds of the blood pressure value and the blood glucose value may also be set in advance. After collecting the body feature value information, the wearable device compares the body feature value with the preset threshold to determine whether the body feature value exceeds the preset threshold range. According to an actual requirement, the wearable device may collect several body feature values of the user at the same time, or just collect one body feature value of the user.

It should be noted that, when the value of the distance between the wearable device and the user is less than or equal to the preset distance threshold, the body feature value of the user is detected. If the value of the distance between the wearable device and the user exceeds the preset distance threshold, it is directly determined that the wearable device is not worn by the user. Certainly, if the body feature value of the user exceeds the preset threshold of the body feature value of the user, it is determined that the wearable device is not worn by the user.

According to the method for detecting a wearable device, and the wearable device in this embodiment of the present invention, a value of a distance between the wearable device and a user may be first detected, then if the value of the distance between the wearable device and the user does not exceed a preset distance threshold, whether a body feature value of the user exceeds a threshold range is detected, and if the body feature value of the user does not exceed the threshold range, that the user has worn the wearable device is determined. That the user has worn the wearable device is determined by combining the value of the distance between the wearable device and the user and the body feature value of the user, so that whether the wearable device is worn on a human body can be identified accurately, and detection accuracy is high. In addition, in this embodiment of the present invention, the value of the distance between the wearable device and the user is first detected, and the body feature value is not detected at the same time, thereby avoiding that a detection time is prolonged when the value of the distance and the body feature value are detected at the same time. Moreover, if the value of the distance between the wearable device and the user exceeds the preset distance threshold, there is no need to detect the body feature value of the user, thereby optimizing a detection process. Therefore, compared with the prior art, in this embodiment of the present invention, whether the user has worn the wearable device can be determined more quickly and more accurately.

It should be understood that, the first execution unit and the second execution unit do not indicate a sequenced relationship, but are to differentiate different execution units. Similarly, a first detection unit and a second detection unit are to indicate different detection units, and details are not described herein.

A person of ordinary skill in the art may understand that, each aspect of the present invention or a possible implementation manner of each aspect may be specifically implemented as a system, a method, or a computer program product. In addition, each aspect of the present invention or the possible implementation manner of each aspect may take a form of a computer program product, where the computer program product refers to computer-readable program code stored in a computer-readable medium.

The computer-readable medium may be a computer-readable data medium or a computer-readable storage medium. The computer-readable storage medium includes but is not limited to an electronic, magnetic, optical, electromagnetic, infrared, or semi-conductive system, device, or apparatus, or any appropriate combination thereof, such as a random access memory (RAM), a read-only memory (ROM), an erasable programmable read only memory (EPROM or flash memory), an optical fiber, and a compact disc read only memory (CD-ROM).

A processor in a computer reads computer-readable program code stored in a computer-readable medium, so that the processor can perform a function and an action specified in each step or a combination of steps in a flowchart; an apparatus is generated to implement a function and an action specified in each block or a combination of blocks in a block diagram.

All computer-readable program code may be executed on a user computer, or some may be executed on a user computer as a standalone software package, or some may be executed on a local computer of a user while some is executed on a remote computer, or all the code may be executed on a remote computer or a server. It should also be noted that, in some alternative implementation solutions, each step in the flowcharts or functions specified in each block in the block diagrams may not occur in the illustrated order. For example, two consecutive steps or two blocks in the illustration, which are dependent on an involved function, may in fact be executed substantially at the same time, or these blocks may sometimes be executed in reverse order.

Obviously, a person skilled in the art can make various modifications and variations to the present invention without departing from the spirit and scope of the present invention. The present invention is intended to cover these modifications and variations provided that they fall within the scope of protection defined by the following claims and their equivalent technologies.

1. A method for detecting a wearable device, the method comprising:
detecting a value of a distance between the wearable device and a user;
determining whether the value of the distance between the wearable device and the user exceeds a preset distance threshold;
if the value of the distance between the wearable device and the user does not exceed the preset distance threshold, detecting a body feature value of the user; and
if the body feature value of the user does not exceed a preset threshold range of the body feature value of the user, determining that the user has worn the wearable device.

2. The method according to claim 1, wherein after the determining that the user has worn the wearable device, the method further comprises:
setting a preset time period; and
detecting the body feature value of the user according to the preset time period.

3. The method according to claim 2, wherein the preset time period is set by the user.

4. The method according to claim 1, wherein the method further comprises:
if the value of the distance between the wearable device and the user exceeds the preset distance threshold, determining that the wearable device is not worn by the user.

5. The method according to claim 1, wherein the method further comprises:
if the body feature value of the user exceeds a preset threshold of the body feature value of the user, determining that the wearable device is not worn by the user.

6. The method according to claim 1, wherein the body feature value of the user comprises: one or more of a temperature value, a heart rate value, a blood glucose value, a blood pressure value, or a blood flow velocity value.

7. The method according to claim 1, wherein the value of the distance between the wearable device and the user is obtained by using a distance sensor.

8. The method according to claim 1, wherein the body feature value of the user is obtained by using a human body feature sensor.

9. A wearable device, comprising:
a distance sensor configured to detect a value of a distance between the wearable device and a user;
a human body feature sensor configured to detect a body feature value of the user; and
a processor configured to:
determine whether the value of the distance between the wearable device and the user exceeds a preset distance threshold,
if the value of the distance between the wearable device and the user does not exceed the preset distance threshold, control the human body feature sensor to detect the body feature value of the user, and
if the body feature value of the user does not exceed a preset threshold range of the body feature value of the user, determine that the user has worn the wearable device.

10. The wearable device according to claim 9, wherein the processor is further configured to:
set a preset time period; and
detect the body feature value of the user according to the preset time period.

11. The wearable device according to claim 10, wherein the preset time period is set by the user.

12. The wearable device according to claim 9, wherein the processor is further configured to:
if the value of the distance between the wearable device and the user exceeds the preset distance threshold, determine that the wearable device is not worn by the user.

13. The wearable device according to claim 9, wherein the processor is further configured to:
if the body feature value of the user exceeds a preset threshold of the body feature value of the user, determine that the wearable device is not worn by the user.

14. The wearable device according to claim 9, wherein the body feature value of the user comprises: one or more of a temperature value, a heart rate value, a blood glucose value, a blood pressure value, or a blood flow velocity value.
